(19) United States
(12) Patent Application Publication
(10) Pub. No.: US 2026/0257917 A1
(43) Pub. Date: Sep. 3, 2026

(54) Stabilizing Fluorophosphorus Compounds at Atmospheric Pressure in Anhydrous Organic Nitrile Liquid Solutions

(71) Applicant: American Hyperform, Inc., Philadelphia, PA (US)

(72) Inventor: William Novis Smith, Philadelphia, PA (US)

(73) Assignee: American Hyperform, Inc., Philadelphia, PA (US)

(21) Appl. No.: 19/068,388

(22) Filed: Mar. 3, 2025

(51) Int. Cl. C01B 25/10; C01B 25/455; C01D 15/00; C07C 17/16; C07C 17/18

(52) U.S. Cl. CPC C01B 25/10; C01B 25/455; C01D 15/005; C07C 17/16; C07C 17/18; C07C 2601/14

(57) ABSTRACT

Organic nitrile solvents unexpectedly provide stable and storable anhydrous solutions of the reactive fluorophosphorus compounds phosphorus pentafluoride (PPF), phosphorus oxytrifluoride (POF₃) (POFL), combinations of PPF with POFL, combinations of PPF with hexafluorophosphoric acid (HPF₆) (HFPPA), and/or combinations of PPF with POFL and HFPPA. The concentration of fluorophosphorus compounds chosen from PPF, HFPPA, and POFL in the organic nitrile solvent by weight is from 5% to 50% at RTP, preferably from 20% to 45% by weight at RTP, and more preferably from 40% to 45% by weight at RTP. Preferred organic nitrile solvents are saturated aliphatic hydrocarbon nitriles or dinitriles. The organic nitrile solution including the fluorophosphorus compounds may be used as a fluorinating agent in the synthesis of pharmaceuticals, battery electrolytes, fluorocarbon refrigerants, and other applications where a stable at RTP liquid phase fluorinating agent is desired for chemical synthesis transformation.

### BACKGROUND

Lithium batteries are used in all major applications for rechargeable and many non-rechargeable battery applications including automotive vehicles, mobile electronic devices, uninterruptable power supplies, robotic devices, and others. A rechargeable lithium-ion battery depends on a cathode (positive electrode), an anode (negative electrode), and an electrolyte that transfers ions between the cathode and anode.

Lithium hexafluorophosphate (LiPF₆) is an important material used as a component of the lithium battery electrolyte that transports ions between the battery anode and cathode. The primary, if not only way that lithium hexafluorophosphate is conventionally made is from the reaction of lithium fluoride with phosphorus pentafluoride (PF₅) (PPF), which may be represented as follows:

LiF(s)+PF₅(g)→LiPF₆(s) where a solvent such as liquid hydrofluoric acid, ethers, and/or esters are used. Additional information regarding this synthesis in the liquid phase using ethers and/or esters may be found in U.S. Pat. No. 3,607,020 to Smith. Three conventional synthesis pathways exist for the formation of PPF which are represented as follows:

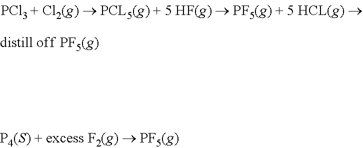

HPF₆(aq)+y SO₃ in H₂SO₄→PF₅(g)+H₂SO₄(aq)→distill off PF₅(g); where y is equivalent to the moles of water present in the aqueous HPF₆ solution.

While lithium fluoride is readily obtainable and is a convenient reagent to use due to its solid form if oxygen and moisture are excluded, PPF is an extremely reactive and corrosive gas having a boiling point of −81° C., thus requiring liquid nitrogen temperatures for condensation and storage at cylinder pressures from 16 to 17 bar. These storage requirements have resulted in PPF being a chemical reagent that is expensive and not readily available for new industrial and synthetic chemistry uses such as in the synthesis of battery electrolytes, pharmaceuticals, and other fluorine containing compounds. Neither can PPF be stabilized by converting it to HPF₆ as is possible for other Group V elements, such as antimony. HF and PPF do not associate to form HPF₆ unless a solvating agent is present, such as at least 25% water or a solvating polar organic solvent such as ethers or esters with or without water.

PPF does not significantly react with the diethyl ether solvent during the duration of the previously described lithium hexafluorophosphate synthesis. However, if PPF is solubilized in the same diethyl ether solvent used for the lithium hexafluorophosphate synthesis reaction and stored at RTP while excluding oxygen and moisture, the PPF will react with the diethyl ether solvent and approximately 10% to 15% of the PPF will be lost after 2-3 months. Additionally, the degradation of the PPF solvated in the ethereal solvent will start to produce highly toxic gaseous hydrogen fluoride (HF) within a few weeks. Hence, while PPF has good solubility in diethyl ether solvents, approaching 25% by weight solubility of the gas in the liquid, PPF cannot be stored in such solvents for much more than a week at room temperature due to an unacceptable rate of degradation and loss of the PPF reagent.

As can be seen from the above description, there is an ongoing need for simple and efficient materials and methods to handle and safely store the PPF reagent. The materials and methods of the present invention overcome at least one of the disadvantages associated with conventional materials and methods of storing and using the PPF reagent.

### SUMMARY

In one aspect, the invention provides a composition for fluorinating organic or inorganic molecules, the composition comprising: an organic nitrile solvent; from 5% to 50% by weight phosphorus pentafluoride; less than 5% degradation impurities of the phosphorus pentafluoride; and less than 1% water by weight. A method of fluorinating an organic or inorganic molecule with the composition is also provided. A method of performing a chemical synthesis transformation by contacting a starting material with the composition is also provided, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.

In another aspect of the invention, a solution consisting essentially of an organic nitrile solvent and from 5% to 45% by weight phosphorus pentafluoride is provided.

In another aspect of the invention, a solution consisting essentially of an organic nitrile solvent and from 5% to 45% by weight phosphorus oxytrifluoride is provided.

In another aspect of the invention, a solution consisting essentially of an organic nitrile solvent and from 3% to 60% by weight hydrogen fluoride is provided.

Other systems, methods, features and advantages of the invention will be, or will become, apparent to one with skill in the art upon examination of the following FIGURES and detailed description. It is intended that all such additional systems, methods, features, and advantages be included within this description, be within the scope of the invention, and be protected by the claims that follow. The scope of the present invention is defined solely by the appended claims and is not affected by the statements within this summary.

### BRIEF DESCRIPTION OF THE FIGURES

The invention can be better understood with reference to the following drawing and description.

FIG. 1 represents the formation of an organic nitrile solution including fluorophosphorus compounds.

### DETAILED DESCRIPTION

Organic nitrile solvents unexpectedly provide stable and storable anhydrous solutions of the reactive fluorophosphorus compounds phosphorus pentafluoride (PF₅) (PPF), phosphorus oxytrifluoride (POF₃) (POFL), combinations of PPF with POFL, combinations of PPF with hexafluorophosphoric acid (HPF₆) (HFPPA), and combinations of PPF with POFL and HFPPA. The concentration of the fluorophosphorus compounds chosen from PPF, HFPPA, and POFL in the organic nitrile solvent by weight is from 5% to 50% at RTP, preferably from 20% to 45% by weight at RTP, and more preferably from 40% to 45% by weight at RTP.

Preferred organic nitrile solvents are saturated aliphatic hydrocarbon nitriles or dinitriles. Preferred organic nitrile solvents include acetonitrile, succinic dinitrile, adipic dinitrile, butyronitrile, propionitrile, isopropionitrile, 2-methyl propionitrile, and cyclohexyl nitrile. More preferred organic nitrile solvents include acetonitrile, propionitrile, and succinic dinitrile. The most preferred at present organic nitrile solvent is acetonitrile (H₃C—C≡N).

The resulting organic nitrile solution including the fluorophosphorus compound or compounds and the organic nitrile solvent is stable. Thus, the solution includes less than 5% degradation impurities for at least 6-months after formation when the solution is stored under anhydrous inert gas in a closed container excluding oxygen and moisture at RTP. In many instances, the solution will achieve less than 3% degradation impurities for at least 6-months.

While not wishing to be bound by any particular theory, the unpredicted solution stability of the fluorophosphorus compounds solvated in the organic nitrile solvent is believed to arise from the unique electron donating ability and polarity of the selected organic nitrile solvents as their polarity is centered around the nitrile functionality. Considering the 16-bar pressure of PPF gas at room temperature and the known instability of organic nitrile solvents at basic pH, the fact that a stable at RTP liquid solvent storage system for gaseous fluorophosphorus compounds at relatively high concentrations of the gaseous fluorophosphorus compounds in the organic nitrile solvent could be formed was unpredicted.

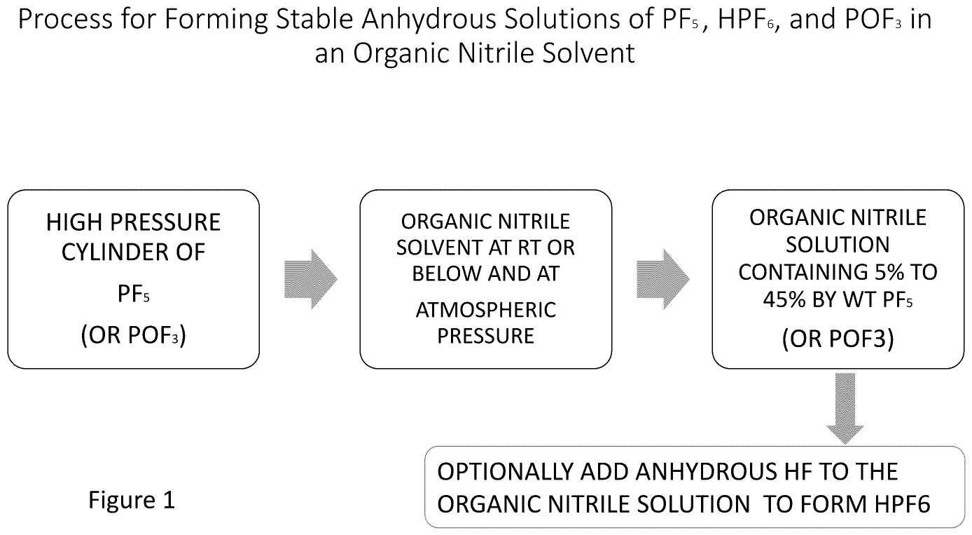

FIG. 1 represents the formation of an organic nitrile solution including fluorophosphorus compounds. The represented organic nitrile solution including the fluorophosphorus compound or compounds and an organic nitrile solvent may be used as a fluorinating agent in the synthesis of pharmaceuticals, battery electrolytes, fluorocarbon refrigerants, and other applications where a liquid phase fluorinating agent is desired for chemical synthesis transformation at or near atmospheric pressure. Preferably, the organic nitrile solution consists essentially of the organic nitrile solvent and the gaseous fluorophosphorus compound and is thus substantially free of impurities.

Selective fluorination of molecules having functional groups such as alcohols, aldehydes, ketones, and other molecules having carbonyl functionality may be performed with the organic nitrile solution to form new carbon-fluorine and other chemical bonds with fluorine.

Once the PPF gas is stabilized in the liquid organic nitrile solvent (ONS), the PPF may be reacted in the liquid phase to form fluorinated compounds, such as those that follow:

1 PF₅ (ONS)+RCHO→RCHF—OPF₄→RCHF₂+POF₃; hence an organic aldehyde is converted to an organic difluoride.

(b) PF₅ (ONS)+RCR′O→RR′CF—OPF₄→RR′CF₂+POF₃; hence an organic ketone is converted to an organic difluoride.

(c) 2 PF₅ (ONS)+RCO₂H→RCF—(OPF₄)₂+HF→RCF₃+2 POF₃; hence an organic carboxylic acid or its salts is converted to an organic trifluoride.

(d) PF₅ (ONS)+RCH₂OH→RCH₂—OPF₄+HF→RCH₂F+POF₃; hence an organic alcohol is converted to an organic fluoride.

(e) PF₅ (ONS)+MF→MPF₆, where M=elements that form fluorides and MPF₆ compounds; hence an inorganic fluoride is converted to a hexafluorophosphate.

(f) PF₅ (ONS)+HF→HPF₆ (ONS); hence an anhydrous hydrogen fluoride is converted to anhydrous hydrogen hexafluorophosphoric acid.

(g) 3 PF₅ (ONS)+(RCO)₂C═O→2 RCF₃+3 POF₃; hence an organic carboxylic acid anhydride is converted to an organic trifluoride.

(h) POF₃ (ONS)+NaOH→NaO₂F₂P+HF; hence an alkali metal hydroxide is converted to an alkali metal difluorophosphate.

In addition to the ability to stabilize PPF, the ability of the organic nitrile solvent to also form and stabilize anhydrous HFPPA was unpredicted, as anhydrous PPF in the presence of anhydrous HF does not form stable HFPPA even at temperatures below −20° C. unless the PPF is combined in an at least 1:1 mole ratio with water, hence in an aqueous solution. However, many inorganic and organic synthetic chemistry reactions do not permit the use of aqueous solutions, thus making the anhydrous organic nitrile solutions advantageous and opening new chemical synthesis transformation pathways that did not previously exist. When desired, HF may be included in the organic nitrile solvent at a weight percent from 0.1% to 8% in relation to the weight of the PPF in the solvent.

In addition to PPF, HFPPA, and POFL, it was also determined that organic nitrile solutions including from 5% to 20% by weight HF were also stable over a 2-year period if maintained under anhydrous inert gas in a closed container at RTP. This was unpredicted because of the known aggressive nature of anhydrous HF toward organic hydrocarbon solvents containing other elements or unsaturated hydrocarbon bonds and especially compounds containing oxygen.

The following examples illustrate one or more preferred embodiments of the invention. Numerous variations may be made to the following examples that lie within the scope of the invention.

### EXAMPLES

### Example 1: Method of Synthesizing and Solvating PPF in Acetonitrile to Form a Stable Solution

A PF₅ gas stream was generated by adding 1 kilogram (kg) of 72% aqueous hexafluorophosphoric acid to a stirred stainless-steel reactor containing 2.5 kg of oleum (65% SO₃ in H₂SO₄) with cooling to maintain the reactor temperature below 15° C. during the reaction. The contents of the reactor were maintained under anhydrous nitrogen and isolated from the atmosphere with an oil bubbler. The PF₅ gas flowing from the reactor was passed through a dry ice in acetone cooled metal condenser to condense out any evolved HF. From the condenser, the purified PF₅ gas was passed into 1 Liter of stirred acetonitrile in a flask maintained at 25° C.

When PF₅ was no longer being fully adsorbed into the acetonitrile and began to pass through the bubbler, the addition of the hexafluorophosphoric acid to the reactor was stopped. The 44% solution of PF₅ in acetonitrile was sealed in a PFA plastic bottle and stored for further use in the following Examples. The 44% solution of PF₅ in acetonitrile was determined to be stable for greater than three years with no appreciable change in appearance, color, or increase in vapor pressure within the bottle.

### Example 2: Formation of a Stable Solution of HPF₆ in Acetonitrile Using HF

0.9 g (0.35 m) of anhydrous HF was added under anhydrous nitrogen to a 44% by weight (0.35 m) solution including 100 g of PF₅ in acetonitrile. A mild exothermic reaction occurred when the anhydrous HF dissolved into the PF₅ acetonitrile solution. The newly formed HPF₆ including acetonitrile solution was stored in a PFA plastic bottle at room temperature for 3 years and showed no discoloration or vapor pressure increase. The anhydrous HPF₆ solution formed in acetonitrile was stable and ready for use as a chemical synthesis reagent after three years.

### Example 3: Formation of Difluoromethyl Benzene from Benzaldehyde in a PF₅ Acetonitrile Solution

35 grams (0.33 m) of benzaldehyde were added to 100 g of PF₅ where the PF₅ was provided as a 44% by weight PF₅ (0.35 m) acetonitrile solution with stirring under an anhydrous inert gas (nitrogen) atmosphere. A mild exothermic reaction occurred when the benzaldehyde dissolved into the PF₅ acetonitrile solution to produce a clear yellow solution. The clear yellow solution was stirred for approximately one hour and then distilled up to a temperature of approximately 130° C. to drive off the formed POF₃, the acetonitrile solvent, and any excess PF₅. The remaining liquid was then fractionally distilled to recover the product. The product, 34 grams of the desired difluoromethyl benzene, was obtained to provide a 78% yield by weight in relation to the benzaldehyde starting material.

### Example 4: Formation of 1,1-Difluoro-Cyclohexane from Cyclohexanone in a PF₅ Acetonitrile Solution

25 grams (0.25 m) of cyclohexanone were added to 100 g of PF₅ where the PF₅ was provided as a 44% by weight PF₅ (0.35 m) acetonitrile solution with stirring under an inert, anhydrous nitrogen atmosphere. A mild exothermic reaction occurred when the cyclohexanone dissolved into the PF₅ acetonitrile solution to produce a clear yellow solution. The clear yellow solution was stirred for approximately one hour and distilled up to a temperature of approximately 95° C. to drive off POF₃ and the acetonitrile solvent. The remaining liquid was then fractionally distilled over a boiling range of 98-102° C. to distill off the desired 1,1-difluoro-cyclohexane product. 28 grams of the desired 1,1-difluoro-cyclohexane were obtained to provide an 88% yield by weight in relation to the cyclohexanone starting material.

### Example 5: Formation of Cyclohexyl Fluoride from Cyclohexanol in a PF₅ Acetonitrile Solution

33 grams (0.33 m) of cyclohexanol were added to 100 g of PF₅ where the PF₅ was provided as a 44% by weight PF₅ (0.35 m) acetonitrile solution with stirring under an inert, anhydrous nitrogen atmosphere. A mild exothermic reaction occurred when the cyclohexanol dissolved into the PF₅ acetonitrile solution to produce a clear solution. The clear solution was stirred for approximately one hour and distilled up to a temperature of approximately 100° C. to remove the acetonitrile solvent and POF₃. The distillation temperature was then increased to a boiling range of 102-105° C. to distill off the desired cyclohexyl fluoride product. 31 grams of the desired cyclohexyl fluoride were obtained to provide an 85% yield by weight in relation to the cyclohexanol starting material.

### Example 6: Formation of Trifluoromethyl Benzene from Benzoic Acid in a PF₅ Acetonitrile Solution

37 grams (0.30 m) of benzoic acid in 60 ml of acetonitrile were added to 200 g of PF₅ where the PF₅ was provided as a 44% by weight PF₅ (0.70 m) acetonitrile solution with stirring under an inert, anhydrous nitrogen atmosphere. A mild exothermic reaction occurred when the benzoic acid dissolved into the PF₅ acetonitrile solution to produce a clear yellow solution. The clear yellow solution was stirred for approximately one hour and distilled up to a temperature of approximately 95° C. to drive off the acetonitrile solvent, excess PF₅, and POF₃. The distillation temperature was then increased to a boiling range of 100-103° C. to distill off the desired trifluoromethyl benzene. 36 grams of the desired trifluoromethyl benzene were obtained to provide an 83% yield by weight in relation to the benzoic acid starting material.

### Example 7: Formation of Lithium Hexafluorophosphate from Lithium Fluoride in a PF₅ Acetonitrile Solution

8.6 grams (0.33 m) of lithium fluoride powder were added to 100 g of PF₅ where the PF₅ was provided as a 44% by weight PF₅ (0.35 m) acetonitrile solution with stirring under an inert, anhydrous nitrogen atmosphere. The resulting white slurry was stirred for about an hour to provide a visually clear solution indicating substantially complete reaction of the lithium fluoride with the PF₅. The solution was filtered to remove any unreacted LiF. The resulting clear solution was concentrated under vacuum at a temperature of 40-45° C. with stirring under an anhydrous inert gas atmosphere. 50 grams of a solid, white lithium hexafluorophosphate powder were recovered to provide a 98% yield by weight in relation to the lithium fluoride starting material.

### Example 8: Formation of a Stable Solution of HF in Acetonitrile

100 grams of anhydrous HF gas were added under anhydrous nitrogen to 100 grams of acetonitrile. A mild exothermic reaction occurred when the HF was dissolved into the acetonitrile solvent. The newly formed stable solution of HF in acetonitrile was stored at room temperature under an anhydrous inert gas atmosphere for one year and showed no discoloration or increase in vapor pressure. The anhydrous HF solution in acetonitrile was determined to be stable and ready for further use as a chemical synthesis reagent after two years.

### Example 9: Formation of a Stable Solution of POF₃ in Acetonitrile

50 grams of phosphorus oxytrifluoride (POF₃) were added under anhydrous nitrogen to 100 grams of acetonitrile. A mild exothermic reaction occurred when the phosphorus oxytrifluoride (POF₃) was dissolved into the acetonitrile. The newly formed stable solution of POF₃ in acetonitrile was stored at room temperature under an anhydrous inert gas atmosphere for one year and showed no discoloration or increase in vapor pressure. The POF₃ solution in acetonitrile was determined to be stable and ready for use as a chemical synthesis reagent after two years.

### Example 10: Formation of a Stable Solution of NaO₂F₂P in Acetonitrile

51 grams of a 33% POF₃ solution in acetonitrile (0.18 m) was added to a slurry of 6.7 grams NaF (0.16 m) in 20 grams of acetonitrile. After stirring for two hours the solution was distilled to dryness under vacuum to produce 18 grams of NaO₂F₂P (99% yield).

### Prophetic Example 1: Method of Synthesizing and Solvating PPF in an Organic Nitrile Solvent Other than Acetonitrile to Form a Stable Solution

A PF₅ gas stream is generated by adding 1 kg of 72% aqueous hexafluorophosphoric acid to a stirred stainless-steel reactor containing 2.5 kg of oleum (65% SO₃ in H₂SO₄) with cooling to maintain the reactor temperature below 25° C. during the reaction. The contents of the reactor are maintained under anhydrous nitrogen and isolated from the atmosphere with an oil bubbler. The PF₅ gas flowing from the reactor is passed through a dry ice in acetone cooled metal condenser to condense out any evolved HF. From the condenser, the purified PF₅ gas is passed into 1 Liter of a stirred organic nitrile solvent chosen from adipic dinitrile, butyronitrile, propionitrile, isopropionitrile, 2-methyl propionitrile, and cyclohexyl nitrile held in a flask maintained at 25° C. When PF₅ is no longer being fully adsorbed into the organic nitrile solvent and begins to pass through the bubbler, addition of the hexafluorophosphoric acid to the reactor is stopped. The 44% solution of PF₅ in the organic nitrile solvent is sealed in a PFA plastic bottle and stored for further use in the preceding Examples 2-7 similarly to the acetonitrile solution of Example 1. The 40-45% solution by weight of PF₅ in the organic nitrile solvent is expected to be stable for at least 6 months and more likely for greater than three years with no appreciable change in color or increase in vapor pressure within the bottle.

### Prophetic Example 2: Solvating PPF in Succinic Dinitrile and Acetonitrile to Form a Stable Solution

227 grams of PF₅ gas is passed into a solution including 100 grams succinic dinitrile and 25 grams acetonitrile with stirring and cooling at 25° C. under an anhydrous inert gas atmosphere. The resulting clear and colorless solution weighs 227 grams, is bottled in a PFA plastic bottle under the anhydrous inert gas, and is stored for 12 months at room temperature. The solution of PF₅ in the combination of succinic dinitrile and acetonitrile solvent system is expected to be stable for at least 6-months and more likely for greater than three years with no appreciable change in color or increase in vapor pressure within the bottle.

### Prophetic Example 3: Formation of a Stable Solution of HPF₆ in an Organic Nitrile Solvent Other than Acetonitrile Using HF

0.9 g (0.35 m) of anhydrous HF is added under anhydrous nitrogen to a 44% by weight (0.35 m) solution including 100 g of PF₅ in an organic nitrile solvent chosen from adipic dinitrile, butyronitrile, propionitrile, isopropionitrile, 2-methyl propionitrile, and cyclohexyl nitrile. A mild exothermic reaction occurs when the anhydrous HF dissolves into the PF₅ solution. The newly formed HPF₆ solution is stored in a PFA plastic bottle at room temperature for 3 years and shows no discoloration or vapor pressure increase. The anhydrous HPF₆ solution is stable and ready for use as a chemical synthesis reagent after three years.

### Prophetic Example 4: Formation of a Stable Solution of HPF₆ in Succinic Dinitrile and Acetonitrile Using HF

0.9 g (0.35 m) of anhydrous HF is added under anhydrous nitrogen to a solution including 100 grams succinic dinitrile, 25 grams of acetonitrile, and 100 g of PF₅. A mild exothermic reaction occurs when the anhydrous HF dissolves into the PF₅ solution. The newly formed HPF₆ solution is stored in a PFA plastic bottle at room temperature for 3 years and shows no discoloration or vapor pressure increase. The anhydrous HPF₆ solution is stable and ready for use as a chemical synthesis reagent after three years.

### Prophetic Example 5: Formation of a Stable Solution of HF in an Organic Nitrile Solvent Other than Acetonitrile

100 grams of anhydrous HF gas is added under anhydrous nitrogen to 100 grams of an organic nitrile solvent chosen from adipic dinitrile, butyronitrile, propionitrile, isopropionitrile, 2-methyl propionitrile, and cyclohexyl nitrile. A mild exothermic reaction occurs when the HF is dissolved into the organic nitrile solvent. The newly formed stable solution of HF in the organic nitrile solvent is stored at room temperature under an anhydrous inert gas atmosphere for one year and shows no discoloration or increase in vapor pressure. The anhydrous HF solution in the organic nitrile solvent is determined to be stable and ready for further use as a chemical synthesis reagent after two years.

### Prophetic Example 6: Formation of a Stable Solution of HF in Succinic Dinitrile and Acetonitrile

100 grams of anhydrous HF gas is added under anhydrous nitrogen to 100 grams succinic dinitrile and 25 grams of acetonitrile. A mild exothermic reaction occurs when the HF is dissolved into the solvent system. The formed stable solution of HF is stored at room temperature under an anhydrous inert gas atmosphere for one year and shows no discoloration or increase in vapor pressure. The HF solubilized in the solvent system is determined to be stable and ready for further use as a chemical synthesis reagent after two years.

### Prophetic Example 7: Formation of a Stable Solution of POF₃ in an Organic Nitrile Solvent Other than Acetonitrile

50 grams of phosphorus oxytrifluoride (POF₃) is added under anhydrous nitrogen to 100 grams of an organic nitrile solvent chosen from adipic dinitrile, butyronitrile, propionitrile, isopropionitrile, 2-methyl propionitrile, and cyclohexyl nitrile. A mild exothermic reaction occurs when the phosphorus oxytrifluoride (POF₃) is dissolved into the organic nitrile solvent. The newly formed stable solution of POF₃ in the organic nitrile solvent is stored at room temperature under an anhydrous inert gas atmosphere for one year and shows no discoloration or increase in vapor pressure. The POF₃ solution in the organic nitrile solvent is determined to be stable and ready for use as a chemical synthesis reagent after two years.

### Prophetic Example 8: Formation of a Stable Solution of POF₃ in Succinic Dinitrile and Acetonitrile

50 grams of phosphorus oxytrifluoride (POF₃) is added under anhydrous nitrogen to 100 grams succinic dinitrile and 25 grams of acetonitrile. A mild exothermic reaction occurs when the phosphorus oxytrifluoride (POF₃) is dissolved into the solvent system. The newly formed stable solution of POF₃ is stored at room temperature under an anhydrous inert gas atmosphere for one year and shows no discoloration or increase in vapor pressure. The POF₃ solubilized in the solvent system is determined to be stable and ready for use as a chemical synthesis reagent after two years.

### Prophetic Example 9: Formation of NaO₂F₂P in an Organic Nitrile Solvent Other than Acetonitrile

51 grams of a 33% POF₃ solution in an organic nitrile solvent chosen from adipic dinitrile, butyronitrile, propionitrile, isopropionitrile, 2-methyl propionitrile, and cyclohexyl nitrile (0.18 m) is added to a slurry of 6.7 grams NaOH (0.17 m) in 20 grams of an organic nitrile solvent chosen from adipic dinitrile, butyronitrile, propionitrile, isopropionitrile, 2-methyl propionitrile, and cyclohexyl nitrile. After stirring for two hours the solution is distilled to dryness under vacuum to produce 15 g of solid NaO₂F₂P.

To provide a clear and more consistent understanding of the specification and claims of this application, the following definitions are provided.

The terms “a”, “an”, and “the” used in the specification claims are to be construed to cover both the singular and the plural, unless otherwise indicated or contradicted by context. No language in the specification should be construed as indicating any non-claimed element to be essential to the practice of the invention.

Unless otherwise indicated, all numbers expressing quantities of ingredients, properties such as amounts, percentages, and the like used in the specification and claims are to be understood as indicating both the exact values as shown and as being modified by the term “about”. Thus, unless indicated to the contrary, the numerical values of the specification and claims are approximations that may vary depending on the desired properties sought to be obtained and the margin of error in determining the values. At the very least, and not as an attempt to limit the application of the doctrine of equivalents to the scope of the claims, each numerical parameter should at least be construed considering the margin of error, the number of reported significant digits, and by applying ordinary rounding techniques.

Unless the context clearly dictates otherwise, where a range of values is provided, each intervening value to the tenth of the unit of the lower limit between the lower limit and the upper limit of the range is included in the range of values.

While the present general inventive concept has been illustrated by description of several example embodiments, and while the illustrative embodiments have been described in detail, it is not the intention of the Applicant to restrict or in any way limit the scope of the general inventive concept to such descriptions. Instead, the descriptions and claims herein are to be regarded as illustrative in nature, and not as restrictive, and additional embodiments will readily appear to those skilled in the art upon reading the above description. Additional modifications will readily appear to those skilled in the art. Accordingly, departures may be made from such details without departing from the spirit or scope of applicant's general inventive concept.

The described methods can be performed in any suitable order unless otherwise indicated or contradicted by context.

Room temperature and pressure (RTP) means from 20 to 28° C. at approximately 100 kPa.

Room temperature (RT) means from 20 to 28° C.

Atmospheric pressure means approximately 100 kPa.

Solid means a substance that is not a liquid or a gas at room temperature and pressure. A solid substance may have one of a variety of forms, including a monolithic solid, a powder, a gel, or a paste.

Liquid means a substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Aqueous solutions are solutions that are greater than 15% water by weight.

Anhydrous means no more than 1% water by weight, preferably no more than 0.1% water by weight.

Stable solutions are determined with either F¹⁹ or P³¹ NMR. If either the F¹⁹ or P³¹ NMR spectra shows greater than 5% degradation impurities from changes in the atomic structure of the F¹⁹ or P³¹ atoms within 6-months when stored under an anhydrous inert gas in the organic nitrile solution at RTP, then the solution is not stable. As the described fluorophosphorus compounds each provide a distinct peak in either the F¹⁹ or P³¹ NMR spectra, the amount of other fluoride or phosphorus compounds, respectively, present in the NMR spectra as impurities is readily quantifiable. Impurities in the NMR spectra can originate from impurities originating from formation of the fluorophosphorus compound or from the degradation of the fluorophosphorus compound after the fluorophosphorus compound is solvated into the organic nitrile solution. Hence, to determine if a stable solution is formed, the NMR spectra of the relevant fluorophosphorus compound is taken within a few minutes of solvating the fluorophosphorus compound into the organic nitrile solvent to determine the amount of formation impurities present, and then after 6-months of storage in the organic nitrile solution to determine the amount of degradation impurities, and thus if the amount of degradation impurities is greater than 5% of the amount of the fluorophosphorus compound originally solvated in the organic nitrile solvent. Hence, degradation impurities exclude formation impurities. The stability of HF in the organic nitrile solvent may be likewise determined using F¹⁹ NMR.

Substantially free of impurities refers to an organic nitrile solution including a fluorophosphorus compound or compounds having no more than 5% degradation products of the fluorophosphorus compound or compounds solvated into the organic nitrile solvent as determined by F¹⁹ or P³¹ NMR.

While various aspects of the invention are described, it will be apparent to those of ordinary skill in the art that other embodiments and implementations are possible within the scope of the invention. Accordingly, the invention is not to be restricted except in light of the attached claims and their equivalents.

1. A composition for fluorinating organic or inorganic molecules, the composition comprising:
an organic nitrile solvent;
from 40% to 45% by weight phosphorus pentafluoride;
less than 5% degradation impurities of the phosphorus pentafluoride; and
less than 1% water by weight.

2. The composition of claim 1, where the composition is a solution of the phosphorus pentafluoride in the organic nitrile solvent.

3. The composition of claim 1, where the organic nitrile solvent is chosen from acetonitrile, succinic dinitrile, adipic dinitrile, butyronitrile, propionitrile, isopropionitrile, 2-methyl propionitrile, and cyclohexyl nitrile.

4. The composition of claim 1, where the organic nitrile solvent is chosen from acetonitrile, propionitrile, and succinic dinitrile.

5. The composition of claim 1, where the organic nitrile solvent is acetonitrile.

6. (canceled)

7. (canceled)

8. The composition of claim 1, where the composition comprises less than 3% degradation impurities of the phosphorus pentafluoride.

9. The composition of claim 1, where the composition comprises less than 0.01% water by weight.

10. The composition of claim 1, where the composition is stable at room temperature and pressure under an anhydrous inert gas atmosphere.

11. The composition of claim 5, where the acetonitrile is saturated with the phosphorus pentafluoride.

12. The composition of claim 1, further comprising from 0.1% to 8% by weight hydrogen fluoride in relation to the weight of the phosphorus pentafluoride in the composition.

13. The composition of claim 1, further comprising hexafluorophosphoric acid.

14. The composition of claim 1, further comprising phosphorus oxytrifluoride.

15. The composition of claim 1, the composition comprising a solution consisting essentially of the organic nitrile solvent and the phosphorus pentafluoride.

16.-25. (canceled)

26. A solution consisting essentially of an organic nitrile solvent and from 40% to 45% by weight phosphorus pentafluoride.

27.-44. (canceled)

45. The solution of claim 26, where the organic nitrile solvent is chosen from acetonitrile, succinic dinitrile, adipic dinitrile, butyronitrile, propionitrile, isopropionitrile, 2-methyl propionitrile, and cyclohexyl nitrile.

46. The solution of claim 26, where the organic nitrile solvent is chosen from acetonitrile, propionitrile, and succinic dinitrile.

47. The solution of claim 26, where the organic nitrile solvent is acetonitrile.

48. The solution of claim 26, where the solution consists essentially of 20% to 45% by weight phosphorus pentafluoride.

49. (canceled)

50. A method of performing a chemical synthesis transformation by contacting a starting material with the solution of claim 26, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.

51. A composition for fluorinating organic or inorganic molecules, the composition comprising:
an organic nitrile solvent;
from 5% to 50% by weight phosphorus pentafluoride;
less than 5% degradation impurities of the phosphorus pentafluoride; and
less than 1% water by weight,
where the organic nitrile solvent is acetonitrile, and
where the acetonitrile is saturated with the phosphorus pentafluoride.

52. The composition of claim 51, where the composition is a solution of the phosphorus pentafluoride in the organic nitrile solvent.

53. The composition of claim 51, where the organic nitrile solvent is chosen from acetonitrile, succinic dinitrile, adipic dinitrile, butyronitrile, propionitrile, isopropionitrile, 2-methyl propionitrile, and cyclohexyl nitrile.

54. The composition of claim 51, where the composition comprises from 20% to 45% by weight phosphorus pentafluoride.

55. The composition of claim 51, where the composition is stable at room temperature and pressure under an anhydrous inert gas atmosphere.

56. A method of performing a chemical synthesis transformation by contacting a starting material with the solution of claim 51, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.

57. A composition for fluorinating organic or inorganic molecules, the composition comprising:
an organic nitrile solvent;
from 5% to 50% by weight phosphorus pentafluoride;
less than 5% degradation impurities of the phosphorus pentafluoride;
less than 1% water by weight; and
from 0.1% to 8% by weight hydrogen fluoride in relation to the weight of the phosphorus pentafluoride.

58. The composition of claim 57, where the composition is a solution of the phosphorus pentafluoride in the organic nitrile solvent.

59. The composition of claim 57, where the organic nitrile solvent is chosen from acetonitrile, succinic dinitrile, adipic dinitrile, butyronitrile, propionitrile, isopropionitrile, 2-methyl propionitrile, and cyclohexyl nitrile.

60. The composition of claim 57, where the composition comprises from 20% to 45% by weight phosphorus pentafluoride.

61. The composition of claim 57, where the composition is stable at room temperature and pressure under an anhydrous inert gas atmosphere.

62. A method of performing a chemical synthesis transformation by contacting a starting material with the solution of claim 57, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.

63. A composition for fluorinating organic or inorganic molecules, the composition comprising:
an organic nitrile solvent;
from 5% to 50% by weight phosphorus pentafluoride;
less than 5% degradation impurities of the phosphorus pentafluoride;
less than 1% water by weight; and
hexafluorophosphoric acid.

64. The composition of claim 63, where the composition is a solution of the phosphorus pentafluoride in the organic nitrile solvent.

65. The composition of claim 63, where the organic nitrile solvent is chosen from acetonitrile, succinic dinitrile, adipic dinitrile, butyronitrile, propionitrile, isopropionitrile, 2-methyl propionitrile, and cyclohexyl nitrile.

66. The composition of claim 63, where the composition comprises from 20% to 45% by weight phosphorus pentafluoride.

67. The composition of claim 63, where the composition is stable at room temperature and pressure under an anhydrous inert gas atmosphere.

68. A method of performing a chemical synthesis transformation by contacting a starting material with the solution of claim 63, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.

69. A composition for fluorinating organic or inorganic molecules, the composition comprising:
an organic nitrile solvent;
from 5% to 50% by weight phosphorus pentafluoride;
less than 5% degradation impurities of the phosphorus pentafluoride;
less than 1% water by weight; and
phosphorus oxytrifluoride.

70. The composition of claim 69, where the composition is a solution of the phosphorus pentafluoride in the organic nitrile solvent.

71. The composition of claim 69, where the organic nitrile solvent is chosen from acetonitrile, propionitrile, and succinic dinitrile.

72. The composition of claim 69, where the composition comprises from 20% to 45% by weight phosphorus pentafluoride.

73. The composition of claim 69, where the composition is stable at room temperature and pressure under an anhydrous inert gas atmosphere.

74. A method of performing a chemical synthesis transformation by contacting a starting material with the solution of claim 69, where the chemical synthesis transformation is chosen from converting an organic alcohol to an organic fluoride, converting an organic aldehyde to an organic difluoride, converting an organic ketone to an organic difluoride, converting an organic carboxylic acid and its salts to an organic trifluoride, converting an organic carboxylic acid anhydride to an organic trifluoride, and converting an inorganic fluoride to an inorganic salt of hexafluorophosphate.
